(19) United States
(12) Patent Application Publication
(10) Pub. No.: US 2012/0312699 A1
(43) Pub. Date: Dec. 13, 2012

(54) DIFFERENTIABLE ANALYTICAL TEST STRIP AND TEST METER COMBINATION

(75) Inventors: Graeme WEBSTER, Inverness (GB); Peter TEMPLETON, Muir of Ord (GB); David ELDER, Inverness (GB); Lawrence RITCHIE, Avoch (GB); James MOFFAT, Inverness (GB); Gavin MACFIE, Inverness (GB)

(73) Assignee: Cilag GmbH International, Zug (CH)

(21) Appl. No.: 13/154,875

(22) Filed: Jun. 7, 2011

(51) Int. Cl. G01N 27/28; G01N 33/66

(52) U.S. Cl. 205/792; 204/403.02; 204/401

(57) ABSTRACT

An analytical test strip and test meter combination for use in the determination of an analyte in a bodily fluid sample includes an analytical test strip and a test meter, and a method for determination thereof. The analytical test strip has an electrode, a first electrical contact pad in electrical communication with the electrode and configured to communicate an electrical response of the electrode to the test meter; and a second electrical contact pad in electrical communication with the electrode and configured to communicate an electrical response of the electrode to the test meter should the test meter be in electrical communication with the second electrical contact pad. In addition, the second electrical contact pad has electrical continuity with the first electrical contact pad and the first and second electrical contact pads are disposed in either of first and second predetermined spatial relationships to one another.

### BACKGROUND OF THE INVENTION

1. Field of the Invention

The present invention relates, in general, to medical devices and, in particular, to analytical test strip and test meter combinations and related methods.

2. Description of Related Art

The determination (e.g., detection and/or concentration measurement) of an analyte in a fluid sample is of particular interest in the medical field. For example, it can be desirable to determine glucose, ketone bodies, cholesterol, lipoproteins, triglycerides, acetaminophen and/or HbA1c concentrations in a sample of a bodily fluid such as urine, blood, plasma or interstitial fluid. Such determinations can be achieved using an analytical test strip and test meter combination.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a simplified perspective depiction of an analytical test strip as can be employed with a test meter in an embodiment of the present invention;

FIG. 2 is a simplified exploded perspective view of the analytical test strip of FIG. 1;

FIG. 3 is a simplified depiction of an analytical test strip and test meter combination according to an embodiment of the present invention;

FIG. 4A is a simplified top view of the first conductive layer of an analytical test strip with a first electrical contact pad and a second electrical contact pad in a first predetermined spatial relationship as can be employed in an embodiment of the present invention;

FIG. 4B is a simplified top view of a first conductive layer of an analytical test strip with a first electrical contact pad and a second electrical contact pad in a second predetermined spatial relationship as can be employed in an embodiment of the present invention and uses labels with a ‘, such as 108’, to distinguish the first conductive layer of FIG. 4B from that of FIG. 4A;

FIG. 5A is simplified depiction of the first conductive layer of FIG. 4A in use with a test meter;

FIG. 5B is simplified depiction of the first conductive layer of FIG. 4B in use with a test meter; and

FIG. 6 is a flow diagram depicting stages in a method for determining an analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, analytical test strip and test meter combinations for use in the determination of an analyte (such as glucose) in a bodily fluid sample (e.g., a whole blood sample) according to embodiments of the present invention include an analytical test strip and a test meter. The analytical test strip has at least one electrode, a first electrical contact pad that is in electrical communication with the electrode and configured to communicate an electrical response of the electrode to the test meter, and a second electrical contact pad that is in electrical communication with the electrode and is configured to communicate an electrical response of the electrode to the test meter should the test meter be in electrical communication with the second electrical contact pad. In addition, the second electrical contact pad has electrical continuity with the first electrical contact pad and the first and second electrical contact pads are disposed in either of first and second predetermined spatial relationships to one another. For example, and as described further herein, the first predetermined spatial relationship can be an aligned spatial relationship while the second predetermined spatial relationship can be a staggered spatial relationship.

The test meter has, in general, a test strip receiving module, with first and second electrical connector pins, and a signal processing module configured to distinguish the predetermined spatial relationship of the first and second electrical contact pads of an analytical test strip inserted into the test strip receiving module. Such distinguishing is accomplished by the signal processing module sensing one of electrical continuity between the first and second electrical connector pins via the first and second electrical contact pads and electrical discontinuity between the first and second electrical connector pins via the first electrical contact pad and, thereby, distinguishing the inserted analytical test strip as having either the first or second predetermined spatial relationship.

Such an analytical test strip and test meter “combination” can be considered an analytical test strip and test meter “set” or analytical test strip and test meter “pairing” and the test meter is considered an associated test meter with respect to the analytical test strip. Moreover, since analytical test strips having either the first or the second predetermined spatial relationships can be distinguished (i.e., differentiated) from one another, such analytical test strips are also referred to as differentiable analytical test strips.

Analytical test strip and test meter combinations according to embodiments of the present invention are beneficial in that the analytical test strips can be readily identified as suitable or unsuitable for use by the test meter based on whether the signal processing module senses an electrical continuity or an electrical discontinuity. Such identification beneficially enables the test meter to proceed with analyte determination only when appropriate, thus avoiding potentially improper, erroneous or inaccurate analyte determinations based on the use of unsuitable analytical test strips.

It is envisioned that various commercial markets can be supplied with analytical test strip and meter combinations according to embodiments of the present invention. For example, commercial market “A” can be supplied with analytical test strips that have a predetermined aligned spatial relationship that results in the sensing of electrical continuity, while commercial market “B” can be supplied with analytical test strips that have a predetermined staggered spatial relationship that results in the sensing of electrical discontinuity. In such a scenario, signal processing modules of test meters supplied to users in markets “A” and “B” would be programmed to identify analyte test strips with the appropriate electrical continuity or discontinuity as suitable for use and analyte test strips with inappropriate electrical continuity or discontinuity as unsuitable for use. If an analytical test strip configured for market A where to be inadvertently employed in market B, a market B test meter would determine that the analytical test strip was unsuitable for use and, if desired, display an appropriate message to a user on a display module of the test meter.

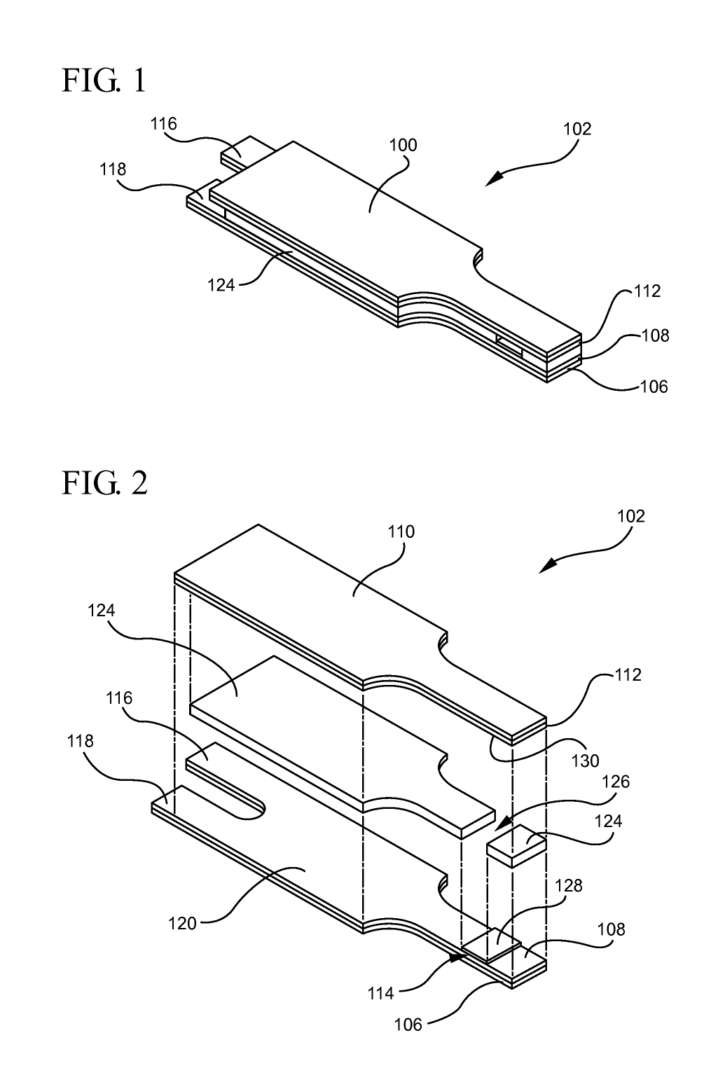

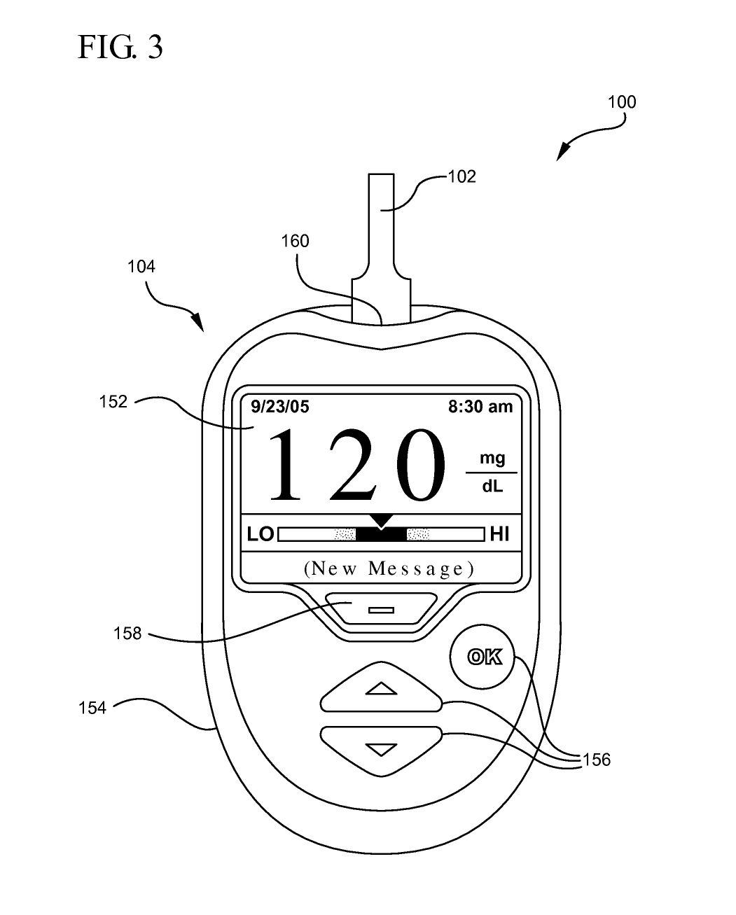

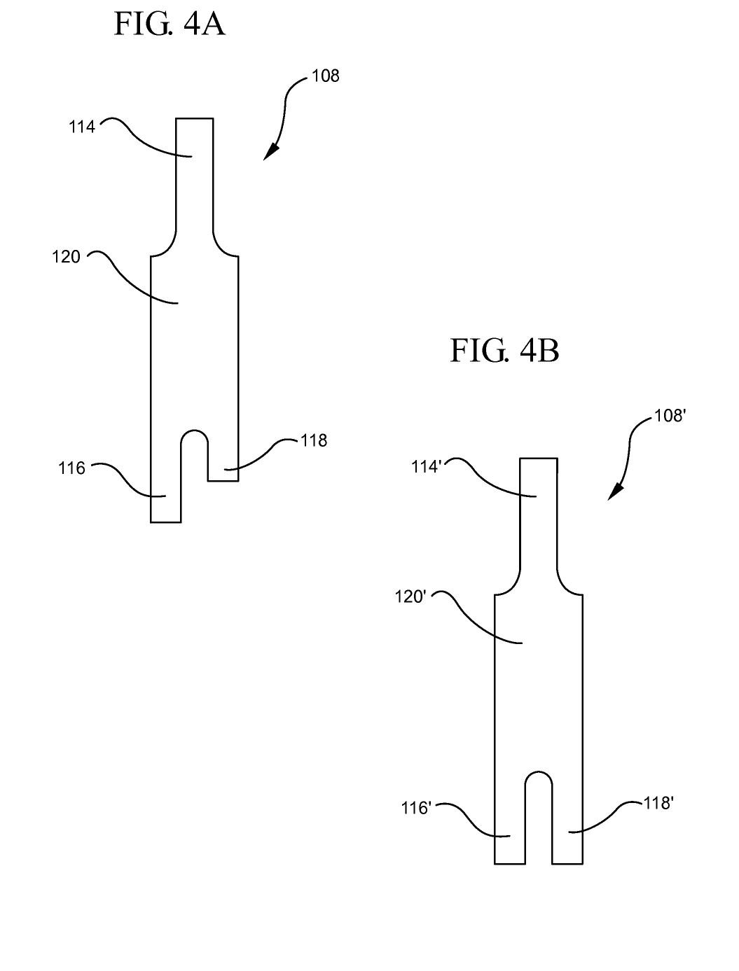

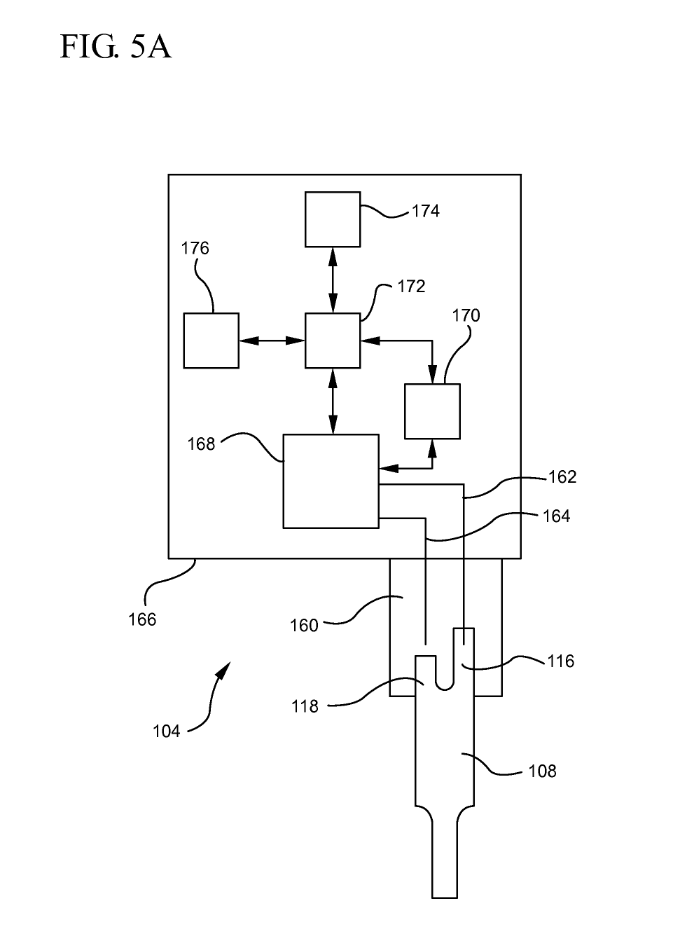

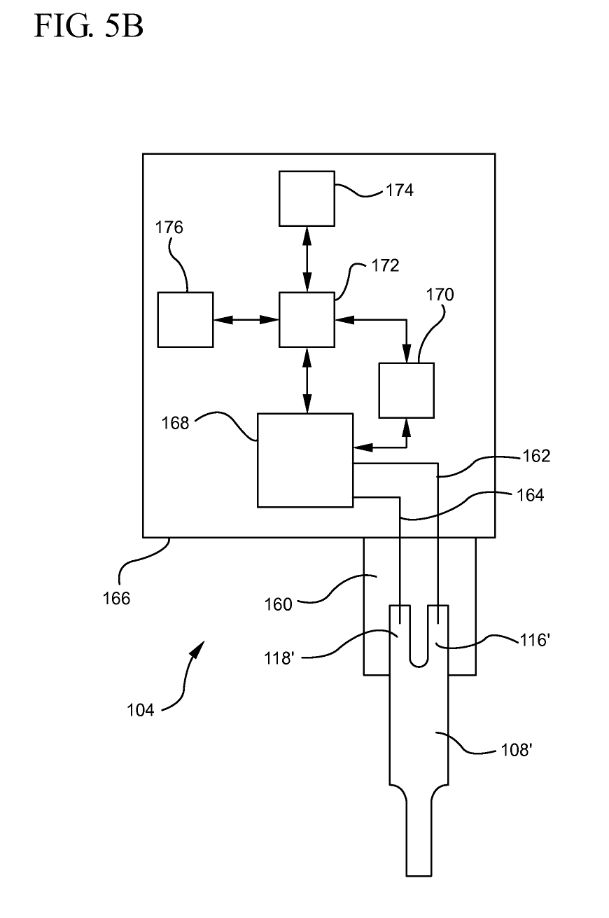

FIG. 1 is a simplified perspective depiction of an analytical test strip as can be employed with a test meter as an analytical test strip and test meter combination according to an embodiment of the present invention. FIG. 2 is a simplified exploded perspective view of the analytical test strip of FIG. 1. FIG. 3 is a simplified depiction of an analytical test strip and test meter combination according to an embodiment of the present invention. FIG. 4A is a simplified top view of a first conductive layer of the analytical test strip of FIG. 1 with a first predetermined spatial relationship. FIG. 4B is a simplified top view of the first conductive layer of an analytical test strip with a second predetermined spatial relationship. FIG. 5A is simplified depiction of the first conductive layer of FIG. 4A in use with the test meter of FIG. 3. FIG. 5B is simplified depiction of the first conductive layer of FIG. 4B in use with the test meter of FIG. 3.

Referring to FIGS. 1-3, 4A, 4B, 5A and 5C, an analytical test strip and test meter combination 100 according to an embodiment of the present invention includes analytical test strip 102 and test meter 104. Analytical test strip 102 includes a first insulating layer 106, with first electrically conductive layer 108 (108′ in FIG. 4B) disposed thereon, and a second insulating layer 110, with second electrically conductive layer 112 disposed thereon. Second insulating layer 110 is disposed above first insulating layer 106.

First electrically conductive layer 108 includes a first electrode 114, first electrical contact pad 116 and second electrical contact pad 118. Analytical test strip 102 also includes connection track 120 that provide electrical communication between first and second electrical contact pads 116 and 118 and first electrode 114. First electrical contact pad 116 and second electrical contact pad 118 are in either of a first predetermined spatial relationship or a second predetermined spatial relationships to one another. Moreover, first electrical contact pads 116 and second electrical contact pad 118 are in electrical continuity due to connection track 120.

FIG. 4A depicts a first predetermined spatial relationship that is referred to as a “staggered” spatial relationship since the first and second electrical contact pads are have unequal extensions along the longitudinal length of the analytical test strip. For example, second electrical contact pad 118 can have an extension of approximately 2.6 mm less than the extension of first electrical contact pad 116. FIG. 4B depicts a first predetermined spatial relationship that is referred to as an “aligned” spatial relationship since the first and second electrical contact pads are have equal extensions along the longitudinal length of the analytical test strip. Once apprised of the present disclosure, one skilled in the art will recognize that the first and second electrical contact pads and predetermined spatial relationships employed in embodiments according to the present invention are not limited to those depicted in FIGS. 4A and 4B. For example, although FIG. 4A depicts second electrical contact pad 118 having a lesser extension than first electrical contact pad 116 this predetermined spatial relationship can be reversed with first electrical contact pad 116 having a lesser extension than second electrical contact pad 118. In addition, although the shape of the first and second electrical contact pads are essentially rectangular in the embodiment of FIGS. 1, 2, 4A-B and 5A-B, the shape of the first and second contact pads can be any suitable shape(s), including complex shapes, that provide for suitable first and second predetermined spatial relationships.

Analytical test strip 102 also includes a patterned spacer layer 124 positioned between second electrically conductive layer 112 and first electrically conductive layer 108. Patterned spacer layer 124 defines a sample-receiving chamber 126 therein. Analytical test strip 102 also includes a reagent layer 128 and second electrically conductive layer 112 includes a second electrode 130, as depicted in FIGS. 1 and 2.

First insulating layer 106 and second insulating layer 112 can be formed, for example, of a plastic (e.g., PET, PETG, polyimide, polycarbonate, polystyrene), silicon, ceramic, or glass material. For example, the first and second insulating layers can be formed from a 7 mil polyester substrate.

First electrode 114, along with second electrode 130 of second electrically conductive layer 112, are configured to electrochemically determine analyte concentration in a bodily fluid sample (such as glucose in a whole blood sample) using any suitable electrochemical-based technique known to one skilled in the art. First electrode 114 can be configured, for example, as a working electrode while second electrode 130 can, for example, be configured as a counter/reference electrode such that analyte test strip 102 is configured as an electrochemical-based analyte test strip.

The first and second conductive layers, 108 and 112 respectively, can be formed of any suitable conductive material such as, for example, gold, palladium, carbon, silver, platinum, tin oxide, iridium, indium, or combinations thereof (e.g., indium doped tin oxide). Moreover, any suitable technique can be employed to form the first and second conductive layers including, for example, sputtering, evaporation, electro-less plating, screen-printing, contact printing, or gravure printing. For example, first conductive layer 108 can be a sputtered palladium layer and second conductive layer 112 can be a sputtered gold layer. A typical but non-limiting thickness for the first and second conductive layers is in the range of 5 nm to 100 nm.

Patterned spacer layer 124 serves to bind together first insulating layer 106 (with conductive layer 108 thereon) and second insulating layer 110 (with second electrically conductive layer 112 thereon), as illustrated in FIGS. 1 and 2. Patterned spacer layer 124 can be, for example, a double-sided pressure sensitive adhesive layer, a heat activated adhesive layer, or a thermo-setting adhesive plastic layer. Patterned spacer layer 124 can have, for example, a thickness in the range of from about 1 micron to about 500 microns, preferably between about 10 microns and about 400 microns, and more preferably between about 40 microns and about 200 microns.

Reagent layer 128 can be any suitable mixture of reagents that selectively react with an analyte such as, for example glucose, in a bodily fluid sample to form an electroactive species, which can then be quantitatively measured at an electrode of analyte test strips according to embodiments of the present invention. Therefore, reagent layer 128 can include at least a mediator and an enzyme. Examples of suitable mediators include ferricyanide, ferrocene, ferrocene derivatives, osmium bipyridyl complexes, and quinone derivatives. Examples of suitable enzymes include glucose oxidase, glucose dehydrogenase (GDH) using a pyrroloquinoline quinone (PQQ) co-factor, GDH using a nicotinamide adenine dinucleotide (NAD) co-factor, and GDH using a flavin adenine dinucleotide (FAD) co-factor. Reagent layer 128 can be formed using any suitable technique.

Test meter 104 includes a display 152, a housing 154, a plurality of user interface buttons 156, an optional soft key 158 and a test strip receiving module 160. Test meter 104 further includes electronic circuitry modules (described with respect to FIGS. 5A and 5B below) within housing 154 for applying a test voltage, and also for measuring a plurality of test current values. Analytical test strip 102 is configured for operative insertion into strip port connector (not shown).

Referring again to FIGS. 5A and 5B in particular, test strip receiving module 160 of test meter 104 includes a first electrical connector pin 162, a second electrical connector pin 164, and a signal processing module 166. First electrical connector pin 162 is configured to contact first electrical contact pad 116 of an analytical test trip, with first electrical contact pad 116 being in electrical communication with a first electrode 114 of the analyte test strip.

Second electrical connector pin 164 is configured to contact second electrical contact pad 118 for the predetermined spatial relationship of FIG. 5B but to not make contact for the predetermined spatial relationship of FIG. 5A.

Once apprised of the present disclosure, one skilled in the art will recognize that embodiments of the present invention can employ more than one electrical connector pin for making electrical contact with electrical contact pad 116. Moreover, embodiments of the present invention can employ more than one electrical connector pin for making electrical contact with electrical contact pad 118 for the predetermined spatial relationship of FIG. 5B, with at least one of the multiple electrical connector pins not making contact in the predetermined spatial relationship of FIG. 5A.

Signal processing module 166 is configured to module is configured to distinguish the predetermined spatial relationship of the first electrical contact pad and the second electrical contact pad of an analytical test strip inserted into the test strip receiving module by sensing one of electrical continuity between the first electrical connector pin and the second electrical connector pins via the first electrical contact pad and second electrical contact pad (as in FIG. 5B) and electrical discontinuity between the first electrical connector pin and the second electrical connector pins via the first electrical contact pad (as in FIG. 5A) and, thereby, distinguish the inserted analytical test strip as having the first predetermined spatial relationship or the second predetermined spatial relationship. Signal processing module 166 can also be configured to determine (using for example software logic) whether the analytical test strip is suitable for use with the test meter or unsuitable for use with the test meter based on the distinguished predetermined spatial relationship.

In the embodiment of FIGS. 5A and 5B, signal processing module 166 includes a test voltage unit 168, a current measurement unit 170, a processor unit 172, a memory unit 174, and a visual display module 176. One skilled in the art will appreciate that the test meter 104 can also include and employ a variety of sensors and circuits that are not depicted in simplified FIGS. 3, 5A and 5B during the distinguishing of an analytical test strip and during determination of an analyte. Moreover, test voltage unit 168, current measurement unit 170, processor unit 172, memory unit 174, and visual display module 176 can also serve to perform additional test meter functions including, for example, the functions described in co-pending U.S. patent application Ser. No. 12/464,935, which is hereby incorporated in full by reference.

Memory unit 174 of test meter 104 includes a suitable algorithm that determines an analyte based on the electrochemical response of analytical test strip 102. The algorithm, therefore, accommodates the electrochemical response of the electrodes within electrochemical-based analytical test strip 10.

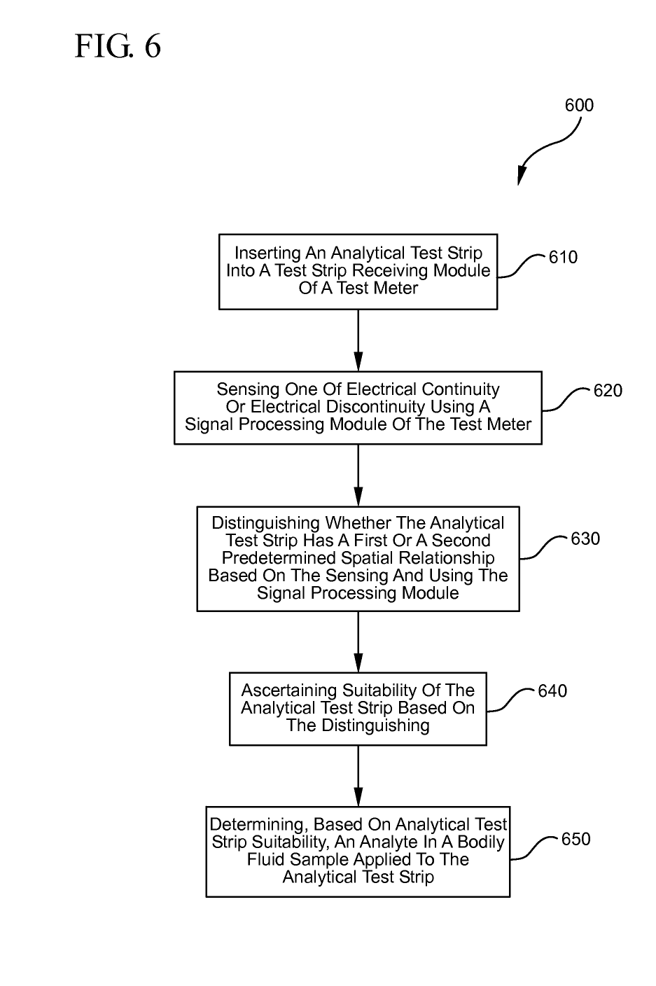

FIG. 6 is a flow diagram depicting stages in a method 600 for determining an analyte (such as glucose) in a bodily fluid sample (for example, a whole blood sample) according to an embodiment of the present invention. Method 600 includes inserting an analytical test strip into a test strip receiving module of a test meter (see step 610 of FIG. 6).

At step 620, a signal processing module of the test meter is used to sense one of (i) electrical continuity between a first electrical connector pin of the test strip receiving module and a second electrical connector pin of the test strip receiving module via a first electrical contact pad and a second electrical contact pad of the analytical test strip and (ii) electrical discontinuity between the first electrical connector pin and the second electrical connector pins via the first electrical contact pad. FIG. 5B (described earlier) illustrates an analytical test strip and test meter combination wherein such electrical continuity would be sensed while FIG. 5A (also described earlier) illustrates an analytical test strip and test meter combination wherein such electrical discontinuity would be sensed.

The signal processing module is used, at step 630 of method 600, to distinguish the inserted analytical test strip as having a first predetermined spatial relationship of the first electrical contact pad and the second electrical contact pad or a second predetermined spatial relationship of the first electrical contact pad and the second electrical contact pad. Non-limiting examples of such first and second predetermined spatial relationships are depicted in FIGS. 4A and 4B, respectively.

Method 600 also includes ascertaining suitability of the analytical test strip based on the distinguishing step (see step 640) and determining, based on analytical test strip suitability, an analyte in a bodily fluid sample applied to the analytical test strip (see step 650).

Once apprised of the present disclosure, one skilled in the art will recognize that methods according to embodiments of the present invention including method 600 can be readily modified to incorporate any of the techniques, benefits and characteristics of analytical test strip and test meter combinations according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. An analytical test strip and test meter combination for use in the determination of an analyte in a bodily fluid sample, the analytical test strip and test meter combination comprising:
an analytical test strip with:
at least one electrode;
a first electrical contact pad in electrical communication with the electrode and configured to communicate an electrical response of the electrode to the test meter; and
a second electrical contact pad in electrical communication with the electrode and configured to communicate an electrical response of the electrode to the test meter should the test meter be in electrical communication with the second electrical contact par, the second electrical contact pad having electrical continuity with the first electrical contact pad; and
a test meter with:
a test strip receiving module with at least a first electrical connector pin and a second electrical connector pin; and
a signal processing module,
wherein:
the first electrical contact pad and the second electrical contact pad are disposed in either of a first predetermined spatial relationship to one another and a second predetermined spatial relationship to one another; and
the signal processing module is configured to distinguish the predetermined spatial relationship of the first electrical contact pad and the second electrical contact pad of an analytical test strip inserted into the test strip receiving module by sensing one of electrical continuity between the first electrical connector pin and the second electrical connector pin via the first electrical contact pad and second electrical contact pad and electrical discontinuity between the first electrical connector pin and the second electrical connector pin via the first electrical contact pad and, thereby, distinguish the inserted analytical test strip as having the first predetermined spatial relationship or the second predetermined spatial relationship.

2. The analytical test strip and test meter combination of claim 1 wherein the first predetermined spatial relationship is an aligned spatial relationship and the second predetermined spatial relationship is a staggered spatial relationship.

3. The analytical test strip and test meter combination of claim 1 wherein the analyte is glucose and the bodily fluid sample is a whole blood sample.

4. The analytical test strip and test meter combination of claim 1 wherein the signal processing module is further configured to determine whether the analytical test strip is suitable for use with the test meter or unsuitable for use with the test meter based on the distinguished predetermined spatial relationship.

5. The analytical test strip and test meter combination of claim 4 wherein the signal processing module is further configured to determine an analyte in a whole blood sample when the analytical test strip is suitable for use with the test meter.

6. The analytical test strip and test meter combination of claim 4 wherein the signal processing module further includes a display module and the signal processing module is further configured to display an error message on the display module when the analytical test strip is unsuitable for use with the test meter.

7. The analytical test strip and test meter combination of claim 4 wherein the sensing of electrical continuity by the signal processing module indicates that the analytical test strip is suitable for use with the test meter and the sensing of electrical discontinuity indicates that the analytical test strip is unsuitable for use with the test meter.

8. The analytical test strip and test meter combination of claim 4 wherein the sensing of electrical discontinuity by the signal processing module indicates that the analytical test strip is suitable for use with the test meter and the sensing of electrical continuity indicates that the analytical test strip is unsuitable for use with the test meter.

9. The analytical test strip and test meter combination of claim 4 wherein the signal processing module employs logic to distinguish the predetermined spatial relationship of the first electrical contact pad and the second electrical contact pad of an analytical test strip inserted into the test strip receiving module.

10. The analytical test strip and test meter combination of claim 9 wherein signal processing module employs software logic to distinguish the predetermined spatial relationship of the first electrical contact pad and the second electrical contact pad of an analytical test strip inserted into the test strip receiving module.

11. A method for determining an analyte in a bodily fluid sample, the method comprising:
inserting an analytical test strip into a test strip receiving module of a test meter;
sensing, using a signal processing module of the test meter, one of electrical continuity between a first electrical connector pin of the test strip receiving module and a second electrical connector pin of the test strip receiving module via a first electrical contact pad and a second electrical contact pad of the analytical test strip and electrical discontinuity between the first electrical connector pin and the second electrical connector pins via the first electrical contact pad;
distinguishing, using the signal processing module, the inserted analytical test strip as having a first predetermined spatial relationship of the first electrical contact pad and the second electrical contact pad or a second predetermined spatial relationship of the first electrical contact pad and the second electrical contact pad; and
ascertaining suitability of the analytical test strip based on the distinguishing step; and
determining, based on analytical test strip suitability, an analyte in a bodily fluid sample applied to the analytical test strip.

12. The method of claim 11 wherein the first predetermined spatial relationship is an aligned spatial relationship and the second predetermined spatial relationship is a staggered spatial relationship.

13. The method of claim 11 wherein the analyte is glucose and the bodily fluid sample is a whole blood sample.

14. The method of claim 11 wherein the signal processing module is further configured to ascertain whether the analytical test strip is suitable for use with the test meter or unsuitable for use with the test meter based on the distinguished predetermined spatial relationship.

15. The method of claim 14 wherein the signal processing module is further configured to determine an analyte in a whole blood sample when the analytical test strip is suitable for use with the test meter.

16. The method of claim 14 wherein the signal processing module further includes a display module and the signal processing module is further configured to display an error message on the display module when the analytical test strip is unsuitable for use with the test meter.

17. The method of claim 14 wherein the sensing of electrical continuity by the signal processing module indicates that the analytical test strip is suitable for use with the test meter and the sensing of electrical discontinuity indicates that the analytical test strip is unsuitable for use with the test meter.

18. The method of claim 14 wherein the sensing of electrical discontinuity by the signal processing module indicates that the analytical test strip is suitable for use with the test meter and the sensing of electrical continuity indicates that the analytical test strip is unsuitable for use with the test meter.

19. The method of claim 14 wherein the signal processing module employs logic to distinguish the predetermined spatial relationship of the first electrical contact pad and the second electrical contact pad of an analytical test strip inserted into the test strip receiving module.

20. The method of claim 19 wherein signal processing module employs software logic to distinguish the predetermined spatial relationship of the first electrical contact pad and the second electrical contact pad of an analytical test strip inserted into the test strip receiving module.
